(19) United States
(12) Patent Application Publication
(10) Pub. No.: US 2012/0076748 A1
(43) Pub. Date: Mar. 29, 2012

(54) Odor conjugater for chemically neutralizing odors

(76) Inventors: Marvin Rembo, Jericho, NY (US); Jaime Rembo, Jericho, NY (US)

(21) Appl. No.: 13/200,617

(22) Filed: Sep. 27, 2011

Related U.S. Application Data
(60) Provisional application No. 61/404,061, filed on Sep. 27, 2010.

(51) Int. Cl. A61L 9/01

(52) U.S. Cl. 424/76.21; 424/76.1

(57) ABSTRACT

An odor conjugater for chemically neutralizing odors.

### RELATED APPLICATIONS

This application claims priority to provisional patent application Ser. No. 61/404,061 filed Sep. 27, 2010.

### FIELD OF THE INVENTION

The present invention relates to an odor conjugater for chemically neutralizing odors.

### BACKGROUND OF THE INVENTION

U.S. Pat. No. 5,031,578 relates to an animal litter box system which reduces odor and extends the useful life of litter by delivering and concentrating urine in an area specifically designed to prevent odor formation. The protective member includes means for forming an opening having a predetermined periphery in its uppermost surface when the protective member is lifted from the litter box to initiate the filtering operation. The protective member further includes liquid sorbent means including an odor abatement agent to prevent or reduce the development of odor in the absorbed liquid.

U.S. Pat. No. 5,290,547 relates to a macroemulsion composition for controlling odors and including droplets of water-immiscible, odor counteracting material dispersed as the discontinuous phase of the macroemulsion in a water soluble binder, which constitutes the continuous phase of the macroemulsion. Upon deposition of the composition onto an appropriate substrate, such as pet litter, and after evaporation of the water within the binder solution, the water soluble binder solidifies on the substrate. In so doing, the solidified water soluble binder substantially encloses the droplets of the odor counteracting material. The water soluble binder easily dissolves upon contact with water in pet excreta, thereby releasing the odor counteracting material in an amount appropriate to counteract the offensive odors at the offensive area where such counteraction is needed most.

U.S. Pat. No. 5,507,250 relates to the addition of urease negative bacteria to sodium smectite clay minerals in pet litter inhibits growth of urease positive bacteria for a period of several days, thereby retarding formation of ammonia and other obnoxious odors. Approximately fifty percent sodium bentonite in the litter causes the litter to clump upon wetting, maintaining the urea in contact with the treated clay and also serving as a buffer to favor growth of the urease negative bacteria.

U.S. Pat. No. 5,819,688 relates to a pet animal odor absorbing mat comprising a mat or pad formed of a fibrous material, such as a cellulosic material, and which is integrally incorporated with a zeolite mineral during the manufacture of the mat. Wood fibers are mixed with a binding agent such as an acrylic latex or other animal non-toxic binder, and the zeolite while in somewhat of a semi-viscous and semi-liquid state and then allowed to harden. The mat is highly effective for use as a bottom layer in cat litter boxes to absorb both urine or other wetness and highly effective, in literally entrapping odors, such as those odors arising from pet animals. The mat thereupon simultaneously releases oxygen. This type of mineral has been found to be particularly effective in removing those odors arising both from the animal urine, as well as other animal excreta.

U.S. Pat. No. 6,077,318 relates to an aqueous composition for reducing malodor impression. The composition comprises from about 0.01% to about 1%, by weight of the composition, of perfume wherein the perfume preferably comprises ingredients having a Clog P of 3 or smaller. Optionally, but preferably, the composition comprises from about 0.1% to about 5%, by weight of the composition of, water-soluble cyclodextrin, from about 0.1% to about 10%, by weight of the composition, of water-soluble metallic salt, from about 0% to about 3%, by weight of the composition, of solubilizing aid. The composition is essentially free of any material that would soil or stain fabric and contains less than about 5%, by weight of the composition of low molecular weight monohydric alcohols.

U.S. Pat. No. 6,682,694 relates to a stable, aqueous odor-absorbing solution, for use on inanimate surfaces. The solution comprises from about 0.1% to about 5%, by weight of the solution, of solubilized, water-soluble, uncomplexed cyclodextrin, an effective amount of a solubilized, water-soluble, antimicrobial preservative having a water-solubility of greater than about 0.3%, optional perfume, and an aqueous carrier. The solution is essentially free of any material that would soil or stain fabric and has a pH of greater than about 3. The solution can be incorporated into a spray dispenser to create an article of commerce that can facilitate treatment of articles and/or surfaces with uncomplexed cyclodextrin solutions of a level that is effective yet is not discernible when dried on the surface.

U.S. Pat. No. 7,229,612 relates to compositions (deodorizing or not) for human and animal excrement, especially pest and livestock, comprised of acidic agents and water soluble polymers, wherein the acidic agents are base neutralizers for the ammonia and indolic amines in the excrement and the water soluble polymers are barrier forming agents for the vapor of the offensive odor producing compounds in the excrement and for slowing down the air oxidative and enzymatic nitrification of the excrement ammonia and organic nitrogen thus dispersing the attenuating nitrate concentrations in the environment. The invention further relates to a method of applying said compositions to excrement. Said compositions may be applied to excrement in liquid form by mixing the liquid composition together with the excrement, or in the form of a spray, by spraying the composition directly onto the excrement.

US patent publication 20020054919 relates to novel compositions and methods for reducing odor. The composition comprises at least one synthetic zeolite, at least one acid and at least one substance selected from a metal oxide, metal, or salt of a metal or metal oxide. A wide range of odors, including ammonia and sulfurous odors, may be controlled by contacting an effective amount of the above composition with the article, substance or environment that emits the undesired odor. The composition may also be incorporated into other substances for odor reduction or control to form odor-controlled articles.

US patent publication 20040016410 relates to a method for neutralizing the odor of low molecular weight thiols, such as found in skunk spray, in an affected area. An effective amount of a cleaning composition including an oil phase having at least one of a hydrocarbon oil and a hydrocarbon solvent, and a water phase containing water and a base is applied to the affected area. The affected area is shampooed with the cleaning composition thereby neutralizing the offensive odor.

US patent publication 2004003779 relates to a method and composition for suppressing residential malodor, said composition comprising a fragrance having at least one perfume material which satisfies one of the following criteria, (1) contains a phenyl ring moiety and has an air diffusion coefficient of >5.7, or (2) contains a C-5 ring moiety, which also contains at least 1 carbon which is sp2 hybridized, and has an air diffusion coefficient of >4.4.

US patent publication 20060045861 relates to a reduced odor composition is composed of at least one cationic polygalactomannan or a derivative of cationic polygalactomannan having a weight average molecular weight (Mw) having a lower limit of 5,000 and an upper limit of 200,000, a light transmittance in a 10% aqueous solution of greater than 80% at a light wavelength of 600 nm, a protein content of less than 1.0% by weight of polysaccharide, and a trimethylamine content of less than 25 ppm in a 10% aqueous solution of the polymer. This composition is prepared by treating the polymer with reagents that reduce the molecular weight of the polymer, removing the water-insoluble solid material, and removing odorous components, including trimethylamine (TMA) and other amines and low molecular weight components from the aqueous phase to produce a polymer that when used in a functional system such as household care, personal care or pet care products has reduced or no odor at acidic, neutral, or alkaline pH values.

US patent publication 20060228323 relates to a composition for removing noxious or malodorous ingredients from materials containing the same, the composition comprising an amphoteric compound; solvent; a compound selected from the group consisting of acids, salts of the acids and combinations thereof; and water, and wherein the relative amounts of each of the components present is sufficient to effect the desired removal of the noxious or malodorous ingredients. The composition may be used as an antimicrobial, biocide, smoke reduction agent, and is biodegradable.

### SUMMARY OF THE INVENTION

The present invention relates to an odor conjugater for chemically neutralizing odors. The composition is not a masking agent. Conjugation describes the situation in which two odoriferous compounds chemically bind and eliminate the odor causing compounds. When sniffed together, the two compounds produce the sensation of very little odor, or no odor at all.

It is an object of the present invention, not to replace the odor with another scent.

It is an object of the present invention for the composition to be effective for pet odors.

It is an object of the present invention for the composition to be useful for odors caused by smoking.

It is an object of the present invention for the composition to be effective against smelly acids, amines, mercaptans and thioethers, including hydrogen sulifide and ammonia.

It is an object of the present invention for the composition to be applied through a fine mist in the air.

It is an object of the present invention for the composition to be sprayed directly on the effected areas.

The present invention relates to a composition comprising water, zinc ricinoleate, Ethylenediaminetetraacetic acid (EDTA); amido propyl betain; and a preservative.

It is an object of the present invention for the preservative to be 1,2-benzisothiazoline-3-one.

It is an object of the present invention for the water to be present in an amount of approximately 90-97%.

It is an object of the present invention for the zinc ricinoleate to be present in an amount of less than 1% to approximately 1.5%.

It is an object of the present invention for the Ethylenediaminetetracetic acid (EDTA) to be present in amount of less than 1% to approximately 1.5%.

It is an object of the present invention for amido propyl betain to be present in an amount between less than 1% to approximately 2%.

It is an object of the present invention for the preservative to be present in an amount between less than 1% to approximately 1.5%.

It is an object of the present invention for the composition to contain no enzymes.

It is an object of the present invention for the composition to contain no scents or perfumes.

It is an object of the present invention for the composition to neutralize and eliminate all smells found in the air and also on all water safe fabrics including curtains, carpets, clothing and blankets.

It is an object of the present invention for the composition to be used in litter boxes, cages, pet living and sleeping areas, as well as on hard surfaces, such as wood or plastic.

It is an object of the present invention to apply the composition to a cloth to wipe down an effected area.

It is an object of the present invention for the composition to be applied to a cloth and then used on a pet's coat.

It is an object of the present invention for the composition to be non-toxic.

It is an object of the present invention for the composition to be organic.

It is an object of the present invention for the composition to be biodegradable.

It is an object of the present invention for the composition to be free of alcohol, phenol, and phenol derivatives, phosphates, and phthalates.

It is an object of the present invention for the composition to comprise zinc salt, emulsifiers, preservatives and odor counteractants.

### DETAILED DESCRIPTION OF THE INVENTION

The invention is a composition which comprises an odor conjugater for chemically neutralizing odors. The composition is not a masking agent. The composition is effective for pet odors, odors caused by smoking or other household odors.

The composition is effective against smelly acids, amines, mercaptans and thioethers, including hydrogen sulifide and ammonia.

In an embodiment, the composition is applied through a fine mist in the air. In an embodiment, the composition is sprayed directly on effected areas.

The composition comprises water, zinc ricinoleate, Ethylenediaminetetraacetic acid (EDTA); amido propyl betain; and a preservative.

In an embodiment, the preservative comprises 1,2-benzisothiazoline-3-one.

In an embodiment, the water is present in an amount of approximately 90-97%.

In an embodiment, zinc ricinoleate is present in an amount of less than 1% to approximately 1.5%.

In an embodiment, Ethylenediaminetetraacetic acid (EDTA) is present in amount of less than 1% to approximately 1.5%.

In an embodiment, amido propyl betain is present in an amount between less than 1% to approximately 2%.

In an embodiment, the preservative is present in an amount between less than 1% to approximately 1.5%.

In an embodiment the preservative is 1,2-benzisothiazoline-3-one, and is present in an amount between less than 1% to approximately 1.5%.

The composition contains no enzymes.

In an embodiment, the composition contains no scents or perfumes.

In an embodiment the composition comprises: approximately 95% water, zinc ricinoleate of approximately less than 1%, Ethylenediaminetetraacetic acid (EDTA) of approximately less than 1%, amido propyl betaine of approximately 1.2%, and approximately less than 1% of 1,2-benzisothiazolin-3-one.

1. A composition for chemically neutralizing odors comprising:
water;
zinc ricinoleate;
Ethylenediaminetetraacetic acid (EDTA);
amido propyl betain; and
a preservative.

2. The composition of claim 1 wherein said preservative is 1,2-benzisothiazoline-3-one.

3. The composition of claim 1 wherein said composition is not a masking agent.

4. The composition of claim 1 wherein said composition chemically binds and eliminates odor causing compounds.

5. The composition of claim 1 wherein said composition when bound to an odor causing compound produces sensation of very little odor, or no odor at all.

6. The composition of claim 1 wherein said composition is effective for pet odors.

7. The composition of claim 1 wherein said composition of effective for odors caused by smoking.

8. The composition of claim 1 wherein said composition is effective against smelly acids, amines, mercaptans and thioethers, including hydrogen sulifide and ammonia.

9. The composition of claim 1 wherein said composition is applied through a fine mist in the air.

10. The composition of claim 1 wherein said composition is sprayed directly on effected areas.

11. The composition of claim 1 wherein said water is present in an amount of approximately 90-97%.

12. The composition of claim 1 wherein said zinc ricinoleate is present in an amount of less than 1% to approximately 1.5%.

13. The composition of claim 1 wherein said Ethylenediaminetetraacetic acid (EDTA) is present in amount of less than 1% to approximately 1.5%.

14. The composition of claim 1 wherein said amido propyl betain is present in an amount between less than 1% to approximately 2%.

15. The composition of claim 1 wherein said preservative is present in an amount between less than 1% to approximately 1.5%.

16. The composition of claim 1 wherein said composition contains no enzymes.

17. The composition of claim 1 wherein said composition contains no scents or perfumes.
